(19) United States
(12) Patent Application Publication
(10) Pub. No.: US 2021/0254121 A1
(43) Pub. Date: Aug. 19, 2021

(54) METHOD FOR DETECTING AND/OR IDENTIFYING AT LEAST ONE TARGET MICROORGANISM PRESENT ON A SURFACE

(71) Applicant: BIOMÉRIEUX, Marcy L'Etoile (FR)

(72) Inventors: Jeanne GROSSELIN, Saint Paul Les Romans (FR); Laurent LEBLANC, Genay (FR); Sébastien MENESSIER, Rive De Gier (FR); Jean-Claude RAYMOND, Bessenay (FR); Bosi MAO, Lyon (FR)

(73) Assignee: BIOMÉRIEUX, Marcy L'Etoile (FR)

(21) Appl. No.: 17/251,297

(22) PCT Filed: Jun. 24, 2019
(86) PCT No.: PCT/FR2019/051534

§ 371 Date: Dec. 11, 2020

(30) Foreign Application Priority Data
Jun. 25, 2018 (FR) 1855641

(51) Int. Cl. C12Q 1/02; G01N 1/38; G01N 1/02

(52) U.S. Cl. CPC C12Q 1/02; G01N 2001/028; G01N 1/02; G01N 1/38

(57) ABSTRACT

A method for detecting and/or identifying at least one target microorganism present on a surface, including the following steps: a) depositing, on the surface, a composition including a synthetic, water-soluble film-forming polymer, the composition being a liquid and/or viscous composition or a foam composition; b) drying the composition in order to allow the formation of a polymer film, c) removing, from the surface, the polymer film including the at least one target microorganism; d) dissolving the polymer film with an aqueous diluent in order to form a solution including the at least one microorganism, e) detecting and/or identifying the at least one target microorganism in all or part of the solution, using at least one detection means.

### TECHNICAL FIELD

The present invention concerns the field of microbiological monitoring. More particularly, the present invention relates to a method for detecting and/or identifying at least one target microorganism present on a surface.

### PRIOR ART

Microbiological monitoring is a critical factor, particularly in industrial and hospital settings. In the pharmaceutical, cosmetics and agribusiness industries, diagnostics enable the production of safe products in compliance with international standards and directives. The manufacture of these products requires very stringent controls to guarantee their microbiological quality and their composition. These microbiological monitoring tests are performed throughout the production chain, from the raw material to the finished product, and are used to confirm, for example, the absence of pathogenic bacteria, sterility (no microorganisms must be present), or the non-proliferation of commensal bacteria (normally present in humans and common at low concentrations) beyond a certain threshold. The production environment (air, water, surfaces) is also monitored regularly by diagnostic tests. Regulations require that certain products, such as injectable medicines, be sterile when they are marketed. This guarantee of sterility is provided by tests on the raw materials, the products undergoing manufacture, the production environment and the finished products. The rigor of these controls is an assurance of quality: it guarantees consumer safety.

Different systems or devices have been developed for monitoring air, liquids and surfaces. Several companies have developed culture media that ensure the growth of microorganisms collected directly from contaminated surfaces and allow a direct reading of the number of colonies on the plate. By way of example, mention may be made of Petrifilm® type agar-coated films or Count-Tact® plates. After removing the plate lid, samples are collected by applying manual pressure to the bottom of the plate for an undefined period of time so that the agar medium comes into the closest possible contact with the surface to be sampled. A major disadvantage of this method, known as culture media imprinting, is the introduction of organic material into sensitive areas such as controlled atmosphere areas, which can be a source of contamination. A lack of reproducibility of results can also be observed, chiefly due to variations in contact between the agar medium and the surface to be tested, due to differences in the manual force applied to the plate, which can vary considerably from one operator to another, or even with the same operator during different sampling operations.

Another method for monitoring surfaces is the so-called smear method, which is based on the detachment of microorganisms by rubbing the surface. This action can be performed with a dry or wet wipe or swab. Wet swabbing is a more effective sampling method than dry swabbing, the superiority of wet swabbing being explained by improved substrate/swab contact due to the presence of water. However, wet swabbing has the disadvantage of low efficiency since it requires both detaching bacteria from the substrate by the swab and then releasing the bacteria from the swab into the liquid. In terms of reproducibility, it is rather difficult to standardize a swab whose preparation will depend on the handler.

The robustness of the different sampling methods has a definite impact on the sensitivity and the reproducibility of microbiological monitoring. There is therefore a real need to provide a new method for collecting samples.

### DISCLOSURE OF THE INVENTION

The present invention is intended to solve the technical problems mentioned above. Thus, an objective of the present invention is therefore to provide a process for sampling, on a surface, microorganisms for purposes of their detection without bringing a nutrient medium to the sampling site. This objective is all the more advantageous in areas with a controlled atmosphere.

Another objective of the present invention is to provide a microorganism sampling process with a good recovery rate.

Another objective of the present invention is to provide a reproducible sampling process necessary for quality control monitoring.

These objectives, among others, are achieved by the present invention which relates to a process for detecting and/or identifying at least one target microorganism present on a surface, comprising the following steps:
a) depositing on said surface, a composition comprising a film-forming water-soluble synthetic polymer, said composition being a liquid and/or viscous composition or a foam composition;
b) drying the composition to allow the formation of a polymer film, removing from the surface said polymer film comprising said at least one target microorganism,
d) dissolving the polymer film with an aqueous diluent to form a solution comprising said at least one microorganism,
e) detecting and/or identifying said at least one target microorganism in all or part of the solution using at least one detection means.

In a preferred embodiment the aqueous diluent is a culture medium.

Preferably, the aqueous diluent is a semi-solid culture medium, the polymer film being dissolved by contact with the semi-solid culture medium. Indeed, the semi-solid culture medium consists of a large quantity of free water allowing the polymer film to dissolve on contact. The solution formed is therefore a semi-solid culture medium comprising the dissolved polymer film.

When said composition is liquid and/or viscous, the invention relates to a process for detecting and/or identifying at least one target microorganism present on a surface, comprising the following steps:
a) depositing on said surface, a liquid and/or viscous composition comprising a film-forming water-soluble synthetic polymer,
b) drying said composition to allow the formation of a polymer film,
c) removing from the surface said polymer film comprising said at least one target microorganism,
d) dissolving the polymer film with an aqueous diluent to form a solution comprising said at least one microorganism,
e) detecting and/or identifying said at least one target microorganism in all or part of the solution using at least one detection means.

Thus the liquid or viscous composition has a controlled texture which is sufficiently liquid and/or viscous to be applied to the area to be tested and form a uniform film, but also sufficiently thick to remain on the test area for the drying time and form a film with a suitable thickness.

Its liquid and/or viscous texture also allows it to perfectly follow the surface on which it is applied, whether the surface is smooth or rough. It is recognized that the microbiological testing of certain surfaces, especially those with roughness, is particularly difficult. In the context of the present application, liquid and/or viscous solution means a solution that flows under its own weight at a temperature comprised between 15-25° C.

In a preferred embodiment the composition is a foam composition. The polymer solution is mixed with CO₂ to obtain a foam. The application of a foam generates a physical effect by introducing gas bubbles into the liquid polymer compound. This allows a better recovery of microorganisms on inert surfaces compared with an application of polymer in liquid form. This is particularly advantageous on rough surfaces such as stainless-steel surfaces. The foam returns to a liquid and/or viscous form after deposition very quickly within a few minutes depending on the amount deposited.

The polymers used in the context of the invention have a number of features allowing the desired properties to be obtained. According to the present invention, “film-forming polymer” means a polymer capable of forming, on its own or in the presence of an auxiliary film-forming agent, a continuous film adhering to a substrate. The formed film has good adhesion properties on inert surfaces such as glass, plastic, crystal polystyrene, stainless steel, and good tensile strength properties in order to be removed from the surface and transported.

The role of the polymer according to the invention is to collect microorganisms for purposes of detection. This polymer is biocompatible, little or non-toxic for microorganisms. After being removed from the surface, the polymer film is dissolved in an aqueous diluent and forms a solution which will be analyzed. According to the present invention, the polymer is therefore a water-soluble polymer. Water-soluble polymers are well known and described by the review “Water soluble polymers for pharmaceutical application, Kadajji and Betageri, Polymers, December 2011”.

Their solubility in the aqueous phase depends in particular on the chemical nature of the repeated monomer units and the weight of the polymer. It can of course be related to the pH or to the ionic strength of the solution in which it is dissolved.

According to the present invention, the polymer is a synthetic homopolymer or copolymer. Preferably, the water-soluble synthetic polymer according to the invention is selected from polyethylene, polyvinyl, polyacrylic, polyoxazoline, their derivatives or a mixture of these polymers.

More preferentially, the water-soluble polymer according to the invention is selected from polyethylene oxide (PEO), polyethylene glycol (PEG), polyvinylpyrrolidone (PVP), polyvinyl alcohol (PVA or PVOH), polyacrylic acid (PAA), polyacrylamide, poly(2-hydroxypropyl methacrylamide), poly(2-ethyl-2-oxazoline).

Preferentially, the polymer according to the invention is selected from the following polymers:
polyethylene oxide with an average molecular weight comprised between 100 000 and 8 000 000 g/mol at concentrations comprised between 2 and 30% (m/v)
Polyethylene glycol with an average molecular weight comprised between 600 and 20 000 g/mol at concentrations comprised between 2 and 30% (m/v)
Polyvinylpyrrolidone with an average molecular weight comprised between 10 000 and 1 300 000 g/mol at concentrations comprised between 2 and 30% (m/v)
Polyvinyl alcohol with an average molecular weight comprised between 9 000 and 200 000 g/mol at concentrations comprised between 2 and 60% (m/v) preferentially between 5 and 50% (m/v) even more preferentially between 15 and 35% (m/v)
Polyacrylic acid (PAA) with an average molecular weight comprised between 450 000 and 4 000 000 g/mol at concentrations comprised between 1 and 2% (m/v)
Polyacrylamide with an average molecular weight comprised between 40 000 and 150 000 g/mol at concentrations comprised between 2 and 20% (m/v)
Poly(2-hydroxypropyl methacrylamide) with an average molecular weight comprised between 30 000 and 50 000 g/mol at concentrations comprised between 2 and 30% (m/v)
Poly(2-ethyl-2-oxazoline) with an average molecular weight of 5 000 g/mol at concentrations comprised between 2 and 10% (m/v)

The composition is prepared according to methods known to the person skilled in the art. The polymer is dissolved in a diluent, usually water, then the composition is sterilized by traditional methods such as filtration, autoclaving, gas, irradiation.

Preferably, the composition according to the invention comprises a surfactant. The surfactants used in the context of the invention have a number of features allowing the desired properties to be obtained. For example, the surfactant is soluble in the polymer solution and improves the peeling properties of the film from surfaces such as plastic, crystal polystyrene, glass, 316L stainless steel. Advantageously, the surfactant has a weak effect on the formation of the polymer film, particularly on the drying time. The surfactant can also neutralize the inhibiting action of antiseptics on the microorganisms present on the surface. Preferably, the surfactant is selected from anionic surfactants such as sodium dodecyl sulfate, cholic acid and sodium dicyclohexyl sulfosuccinate, cationic surfactants such as benzalkonium chloride, non-ionic surfactants such as diethylene glycol, polysorbate 80 and saponin, zwitterionic surfactants such as 3-(N,N-dimethyltetradecylammonio)propanesulfonate and 3-[(3-cholamidopropyl)dimethylammonio]-1-propanesulfonate hydrate.

Preferentially the surfactant is polysorbate 80. Advantageously, polysorbate 80 has a concentration equal to or less than 5%, preferentially between 0.5 to 1%.

The composition may also comprise other additives such as neutralizing agents, reducing agents, antioxidant agents in order to improve the recovery of stressed microorganisms. Coloring agents can also be added in order to better visualize the polymer film.

To improve the film-forming properties of the composition, an auxiliary film-forming agent can advantageously be added. Such an auxiliary film-forming agent can be selected from all the compounds known to the skilled person as being capable of fulfilling the desired function, and in particular be selected from the plasticizing agents and coalescing agents of the film-forming polymer(s).

The composition will be deposited and spread with or without an applicator. It can for example be deposited by pipetting and then spread using a sterile loop. When it is a foam composition, the polymer solution is placed in a device containing a gas (for example CO₂, air, nitrogen) in order to create the foam composition by mixing during deposition. The foaming composition reverts to a liquid and/or viscous composition very rapidly after deposition.

The composition according to the invention is then dried to allow the formation of a polymer film. The thickness of the film formed is for example greater than 40 μm, preferably comprised between 100 μm and 500 μm, more preferentially comprised between 100 and 300 μm.

This drying phase can be a physical and/or chemical drying, optionally in the presence of a catalyst. It can be carried out at room temperature, in the open air or under air flow.

Depending on the temperature, the air humidity, the dimensions (size, thickness) of the composition deposited on the surface, said composition will be dried at a minimum until the polymer film is formed. Drying allows the formation of a network within the composition that will capture microorganisms present on the surface. The drying time will be substantially the same whether the composition has been deposited in foamy form or in liquid and/or viscous form.

The polymer film according to the invention is weakly adherent, allowing its removal from the surface without risk of rupture and leaving said surface relatively free of polymer film residue. Once the polymer film is removed from the surface, it can be easily moved from the sampling area to the analysis area without rupturing or tearing. Preferentially, the polymer film has an elastic modulus (G′) greater than 100 000 Pa as measured by an HR2 rheometer (TA Instrument).

Advantageously, the polymer film comprises a surfactant that reduces the force required to remove the film from the substrate to which it adheres.

According to the present invention, the polymer film in which the microorganism is captured is then dissolved in an aqueous diluent to form a solution which will be analyzed. Thus, the process according to the invention has the advantage of dispensing with the step of detaching the microorganisms from the sample substrate, such as the step of desorbing the microorganisms from a swab. The recovery rate, i.e. the ratio between the quantity of microorganisms collected and the quantity present on a surface, is thus particularly optimized. The dissolution step can be accelerated by shaking the solution and the diluent.

Another advantage of the present invention is to use the solution directly for detecting microorganisms. In an advantageous embodiment, the aqueous diluent is a culture medium that will dissolve the polymer film when the polymer film is brought into contact with it. Thus, in this preferred embodiment, the solution is a culture medium comprising the microorganism to be detected.

“Culture medium” means a medium comprising all the elements necessary for the survival and/or growth of microorganisms and, in particular, the microorganisms sought (for example buffered peptone water). The culture medium may contain possible additives, for example:

peptones, one or more growth factors, carbohydrates, one or more selective agents, buffers, one or more gelling agents, one or more vitamins, etc. This culture medium can be in liquid or gelled form ready for use, i.e. ready to be inoculated in tubes, in bottles or on Petri dishes. The expression “culture medium” obviously encompasses enrichment media and broths. Different culture media can be used according to the requirements of the microorganisms to be detected.

The process according to the invention may comprise a step of incubating the solution, before or during the detection step, at a temperature and for a period of time sufficient to allow the growth of said at least one microorganism.

Incubation is generally carried out at a temperature ranging from 20 to 52° C. for a predetermined period of time, for example from 6 h to 28 days.

“Detection means” refers a means of detection which makes it possible to detect and/or measure directly or indirectly one or more biological and/or physicochemical parameters of a biological sample.

A classic detection means used in microbiology consists of solid, liquid or semi-solid culture media that are inoculated. Thus, in a preferred embodiment the detection means is the culture medium in which the polymer film has been dissolved.

Thus, in a preferred embodiment, the process comprises the following steps:
a) depositing on said surface, a liquid and/or viscous composition comprising a film-forming water-soluble synthetic polymer,
b) drying said composition to allow the formation of a polymer film,
c) removing from the surface said polymer film comprising said at least one target microorganism,
d) dissolving the polymer film with a culture medium to form a solution comprising said at least one microorganism,
e) detecting and/or identifying said at least one target microorganism in all or part of the solution using at least one detection means, said detection means being said solution.

In this embodiment, the water-soluble polymer film is dissolved in an aqueous diluent comprising a liquid or semi-solid culture medium. In the case where the film is deposited on a semi-solid culture medium, the water contained in the agar solubilizes the film and it disappears completely from the surface of the culture medium. This inoculated culture medium then constitutes the solution according to the invention. No filtration or separation step to recover the microorganisms is necessary. The culture media are then incubated under the usual culture conditions. The detection and/or identification step is therefore carried out directly from the solution. After incubation, isolated colonies can be easily counted in order to give a quantitative result of the surface monitoring. In this particular embodiment, the culture medium plays the dual role of aqueous diluent and of detection means.

The invention therefore relates to a detection and/or identification process comprising thy: following steps:
a) depositing on said surface, a liquid and/or viscous composition comprising a film-forming water-soluble synthetic polymer,
b) drying said composition to allow the formation of a polymer film,
c) removing from the surface said polymer film comprising said at least one target microorganism,
d) dissolving the polymer film by contact with a culture medium
e) detecting and/or identifying said at least one target microorganism in said culture medium

Preferentially, the culture medium is semi-solid.

In another preferred embodiment, when the composition is a foam composition, the process comprises the following steps:
a) depositing on said surface a foaming composition comprising a film-forming water-soluble synthetic polymer,
b) drying said composition to allow the formation of a polymer film,
c) removing from the surface said polymer film comprising said at least one target microorganism,
d) dissolving the polymer film by contact with a culture medium
e) detecting and/or identifying said at least one target microorganism in said culture medium.

Preferentially, the culture medium is semi-solid.

In another embodiment, the detection and/or identification step consists of a kinetic microorganism detection method by colorimetry such as the BacT/ALERT® technology. The microorganisms present in the medium produce CO₂ during their growth phase. The CO₂ produced induces a decrease in the pH of the culture medium. This change in pH induces the colorimetric shift of a sensor composed of silicone impregnated with liquid emulsion detectors (LES), placed at the bottom of each bottle. An LED sends a light beam on the sensor. A photodiode collects the intensity of light reflected by the sensor in the form of a reflectance unit. The reflectance units are analyzed over time. The polymer film is dissolved in a physiological solution and then injected into a BacT/ALERT® bottle (bioMérieux) for automatic kinetic reading.

In another embodiment, the detection and/or identification step consists of a biochemical colorimetric method miniaturized on a card specifically designed and named Vitek®. The polymer film is placed in a hemolysis tube and then solubilized. The tubes are loaded with a VITEK® card. The system manages the incubation and reading of each card without any other intervention. Results are obtained within 2 to 18 hours after loading.

In another embodiment, the detection and/or identification step consists of a method by flow cytometry or solid phase cytometry. This method consists of individual cell analysis by optical detection.

In another embodiment, the detection and/or identification step consists of a microorganism counting method based on the most probable number (MPN) technique such as the Tempo® technology. This method consists of the analysis of serial dilutions of the initial sample. The Tempo technology miniaturizes this test on a specifically designed card. The polymer film is brought into contact with a dehydrated culture medium contained in a Tempo® bottle (bioMérieux). After rehydration and complete solubilization, the Tempo® cards are filled and then incubated before being automatically read by fluorescence.

In another embodiment, the detection and/or identification step consists of a method for detecting microorganisms by PCR. In the Gene-Up® technology, the polymer film is brought into contact with an enrichment medium. After enrichment, the sample is lysed and brought into contact with PCR reagents. The plate is placed in the Gene-Up® instrument for detection via the PCR technique. Another applicable molecular biology technology may be the Filmarray® technology.

In another embodiment, immunoassays constitute another of the technologies used for the detection test. They make use of the immunogenic characteristics of the microorganisms sought. For example, mention may be made of the VIDAS® detection means. The polymer film is deposited in an enrichment medium tube and incubated. After enrichment, the sample is placed in a VIDAS® strip (bioMérieux).

### BRIEF DESCRIPTION OF THE FIGURES

FIG. 1 is a graph representing on the ordinate the decrease in thickness of the liquid and/or viscous composition during drying (in μm) as a function of the drying time (in hours).

FIG. 2 is an ordinate graph representing the loss modulus (G″) and the elastic modulus (G′) of the liquid and/or viscous composition expressed in pascals as a function of the drying time (in hours).

FIG. 3 is a graph representing on the ordinate the viscosity of the liquid and/or viscous composition expressed in mPa per second as a function of the PVOH polymer concentration expressed in % (m/v).

### DETAILED DESCRIPTION OF THE INVENTION

The following examples will allow the present invention to be better understood. However, these examples are given by way of illustration only and must in no case be regarded as limiting the scope of said invention in any way.

### Example 1a: Preparation of Liquid and/or Viscous Polymer Compositions

Different concentrations of water-soluble polymer were used: from 2 to 50% m/v. Different concentrations of surfactants were used: from 0 to 5% v/v or m/v.
1) The water-soluble polymer is weighed in glass bottles and diluted with demineralized water.
2) The whole is shaken and heated in order to completely dissolve the polymer in solution.
3) If a surfactant is present, it is added to the solution by pipetting (if liquid) or by weighing (if solid).
4) The composition is autoclaved in a liquid cycle, with a plateau at 120° C. for 16 min.
5) The composition is stored at room temperature

### Example 1b: Preparation of Foaming Polymer Compositions

The preparation of foaming compositions differs only by an additional step (step 6) in which a gas is added.
1) The water-soluble polymer is weighed in glass bottles and diluted with demineralized water.
2) The whole is shaken and heated in order to completely dissolve the polymer in solution.
3) If a surfactant is present, it is added to the solution by pipetting (if liquid) or by weighing (if solid).
4) The solution is autoclaved in a liquid cycle, with a plateau at 120° C. for 16 min.
5) The composition is stored at room temperature
6) The composition is added in a device containing CO₂ in order to create the foam composition by mixing during deposition.

### Example 2: Testing of Families of Water-Soluble Polymers

Different families of water-soluble polymers were tested for the following properties:
Dissolution of the polymer in water and obtaining a homogeneous solution
Ability to form a film by drying
Peeling of the film from a surface (crystal polystyrene plastic, glass, 316L stainless steel)
Water-solubility of the film in contact with an aqueous diluent
Detachment and then detection of at least one microorganism

The polymers were prepared according to the following protocol:
1) The polymer is weighed in glass bottles and diluted with demineralized water.
2) The whole is shaken and heated in order to completely dissolve the polymer in solution.
3) A surfactant is added to the solution by pipetting at a concentration of 0.5% v/v.
4) The solution is stored at room temperature

The water-soluble synthetic polymers having demonstrated all the properties described above are preferentially:
Poly(ethylene oxide) (PEO) with an average molecular weight comprised between 100 000 and 8 000 000 g/mol (Sigma Aldrich; item: 181986, 372781, 372838) at concentrations comprised between 2 and 30% (m/v)
Poly(ethylene glycol) (PEG) with an average molecular weight comprised between 600 and 20 000 g/mol at concentrations comprised between 2 and 30% (m/v)
Poly(vinyl pyrrolidone) (PVP) with an average molecular weight comprised between 10 000 and 1 300 000 g/mol (Sigma Aldrich; item: PVP10, PVP360, 437190), at concentrations comprised between 2 and 30% (m/v)
Poly(vinyl alcohol) (PVA or PVOH) with an average molecular weight comprised between 9 000 and 200 000 g/mol (Sigma Aldrich; item: 360627; Merck Millipore; item: 8.43866.1000, 8.43867.1000), at concentrations comprised between 2 and 50% (m/v)
Poly(acrylic acid) (PAA) with an average molecular weight comprised between 450 000 and 4 000 000 g/mol (Sigma Aldrich; item: 181285, 306231), at concentrations comprised between 1 and 2% (m/v)
Poly(acrylamide) with an average molecular weight comprised between 40 000 and 150 000 g/mol (Sigma Aldrich; item: 738743, 749222) at concentrations comprised between 2 and 20% (m/v)
Poly(2-hydroxypropyl methacrylamide) with an average molecular weight comprised between 30 000 and 50 000 g/mol (Sigma Aldrich; item: 804746) at concentrations comprised between 2 and 30% (m/v)
Poly(2-ethyl-2-oxazoline) with an average molecular weight of 5 000 g/mol (Sigma Aldrich; item: 773379) at concentrations comprised between 2 and 10% (m/v)

### Example 3: Test of Families of Surfactants

Different families of surfactants were tested for the following properties:
Dilution of the surfactant in the polymer solution and obtaining a homogeneous solution
Little or no negative impact on the cohesion of the film, the water solubility of the film, the formation of a film by drying.
Facilitates the peeling of the film from a surface (crystal polystyrene plastic, glass, 316L stainless steel)
Detachment and then detection of at least 1 microorganism

Polymer solutions containing a surfactant were prepared according to the following protocol:
1) The polymer is weighed in glass bottles and diluted with demineralized water.
2) The whole is shaken and heated in order to completely dissolve the polymer in solution.
3) The surfactant is weighed (if in solid form) or measured (if in liquid form) and then added to the solution at a concentration of 0.5% m/v or 0.5% v/v.
4) The solution is stored at room temperature

The families of surfactants having demonstrated all the properties described above are preferentially:
Anionic surfactants, such as sodium dodecyl sulfate (Sigma Aldrich, item L3771), cholic acid (Sigma Aldrich, item C1129) and sodium dicyclohexyl sulfosuccinate (Sigma Aldrich, item 86141)
Cationic surfactants, such as benzalkonium chloride (Sigma Aldrich, item 12060)
Non-ionic surfactants, such as diethylene glycol (Sigma Aldrich, item 93171), polysorbate 80 (Acros Organics; item 278630000) and saponin (Sigma Aldrich, item 84510)
Zwitterionic surfactants, such as 3-(N,N-dimethyltetradecylammonio)propanesulfonate (Sigma Aldrich, item 40772) and CHAPS or 3-[(3-cholamidopropyl)dimethylammonio]-1-propanesulfonate hydrate (Sigma Aldrich, item C9426)

### Example 4: Polymer Drying Speed (Base 25 cm²)

In order to evaluate the rate at which the water-soluble polymer composition is capable of changing from a liquid and/or viscous state to a solid state in the form of a film, tests were carried out under various conditions.

The polymer composition 30% m/v PVOH+0.5% v/v polysorbate 80 was manufactured according to Example 1 and then stored at room temperature.

1 ml of solution is deposited on a 316L stainless steel square surface of 5 cm×5 cm, i.e. 25 cm². The deposits were placed in a ventilated oven (30% ventilation) at different temperatures or under a laminar flow hood at room temperature.

The time required for the entire 25 cm² polymer surface to be in film form was timed.

The results are shown in the following table:

| | Laminar flow | 22.5° C. | 32.5° C. | 37° C. |
|---|---|---|---|---|
| Drying condition | hood (RT) | oven | oven | oven |
| Average drying speed | 1.23 | 2.50 | 1.72 | 2.06 |
| (in hours) | | | | |
| Standard deviation | 0.06 | 0.45 | 0.36 | 0.14 |

Conclusion:

It has been shown that the drying speed of water-soluble polymer is more dependent on ventilation than on temperature (better ventilation under a flow hood than in an oven).

The drying speed is comprised between 1.5 h and 3 h, demonstrating the speed with which the polymer goes from a viscous state to a solid state.

### Example 5: Rheological Analysis of Compositions According to the Invention During Drying

Rheological analyses were performed in order to physically characterize the behavior of the polymer solution during drying.

A 10% m/v PVOH+0.5% v/v polysorbate 80 polymer solution and a 10% m/v PVOH+5% v/v polysorbate 80 polymer solution were manufactured according to Example 1 and then stored at room temperature.

The rheological study was performed on a DHR-2 rheometer (TA Instruments) with a lower platform (Peltier) for temperature maintenance and an upper platform covered with a plastic “doughnut”. This doughnut-shaped cover allows a slow and uniform drying of the gel along a radial axis, ensuring a homogeneous gel thickness during drying.

Roughly 1 ml of polymer solution is deposited on the lower platform of the rheometer, then the upper platform is slowly lowered in contact with the solution, to obtain an initial gap of 500 μm. The experiment is performed at a constant temperature of 25° C.

The height of the upper platform is controlled by the rheometer so as to impose on the sample a constant zero force that does not vary during drying of the gel. The height of the sample (gap) can thus be monitored over time.

FIG. 1 is a graph representing the decrease in sample thickness (10% m/v PVOH+5% v/v polysorbate 80 polymer solution) during drying.

The upper platform also applies low amplitude shear oscillations (stress amplitude: y=0.1% and frequency=1 Hz) to measure the viscoelastic properties of the sample during drying.

The viscoelastic properties of the sample are reflected by the viscous modulus or loss modulus (G″) and the elastic modulus or storage modulus (G′). When the elastic modulus becomes higher than the viscous modulus it is possible to determine the critical PVOH concentration corresponding to the gel point.

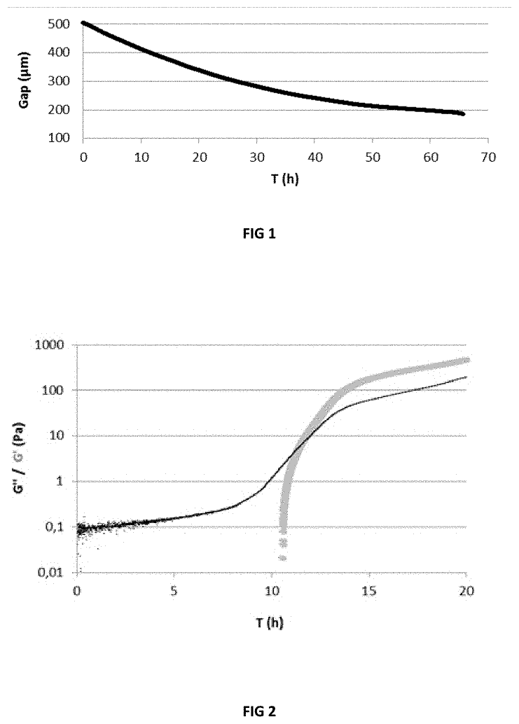

FIG. 2 is a graph showing the change in the viscous (G″) and elastic (G′) moduli during the drying of the 10% m/v PVOH+5% v/v polysorbate 80 polymer solution.

At the gel point, the critical PVOH concentration obtained is comprised between 10 and 20% m/v.

The elastic modulus obtained at the end of drying, and which characterizes the solid aspect of the polymer film, is greater than 100 000 Pa.

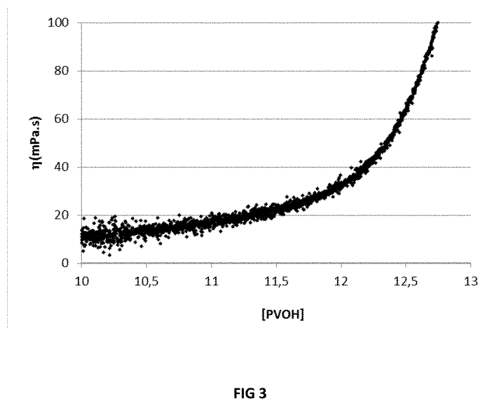

Finally, the viscosity of the liquid and/or viscous composition comprising the polymer was calculated as a function of the PVOH polymer concentration, represented in FIG. 3. This viscosity characterizes the polymer solution in its liquid/viscous form.

### Example 6: Film Peel Force

In order to physically characterize the adhesion of the film to the surfaces, film peel force tests were performed on different surfaces.

The polymer solutions were manufactured according to Example 1 and then stored at room temperature. 1.5 g of solution is deposited on a rectangular surface measuring 6.5 cm by 4 cm (26 cm²) and then dried for 24 hours at room temperature.

After drying and formation of the film, an initial section of about 1 cm of film is peeled off the surface over the width of 4 cm. This initial section is inserted into the clamp of an MTS® System traction bench, at an angle of about 90°, at a distance of 6 mm. The peeling speed is 50 mm/min, over a distance of 50 mm. The average force required to peel off the film is calculated by the traction bench and presented in the table below.

| | | Peel force (in N) | |
|---|---|---|---|
| | | | SD (standard |
| Composition | Surface | AVG | deviation) |
| 30% PVOH, 0.5% polysorbate 80 | Plastic | 0.91 | 0.14 |
| 30% PVOH, 0.5% polysorbate 80 | Glass | 1.99 | 0.82 |
| 30% PVOH, 0.5% polysorbate 80 | Stainless steel | 2.32 | 0.52 |
| 10% PVOH, 5% polysorbate 80 | Plastic | 0.39 | 0.03 |
| 50% PVOH | Stainless steel | 12.72 | 2.34 |

### Example 7: Film Elongation Force

In order to characterize the tear resistance of the film, elongation force tests were carried out.

The polymer solutions were manufactured according to Example 1 and then stored at room temperature. 1 g or 3 g of solution was deposited on a round template with a surface area of 23 cm² and then dried for 24 hours at room temperature. After peeling off the film, a 3 cm×3 cm square was cut in the center of the circle to ensure a homogeneous thickness of the test sample. The cut film is placed between the two clamps of the tensile tester, at a distance of 10 mm. The elongation speed is 10 mm/min until the film breaks. The value selected is the maximum force value required to rupture the film, visible on the elongation curve by a sharp inflection point (sudden drop).

The results are presented in the following table:

| | Cast | Thick- | Elongation force (in N) | |
|---|---|---|---|---|
| Composition | weight | ness | AVG | SD |
| 30% PVOH, 0.5% polysorbate | 1 g | 44 μm | 38.76 | 3.65 |
| 30% PVOH, 0.5% polysorbate | 3 g | 228 μm | 83.45 | 3.17 |
| 10% PVOH, 0.5% polysorbate | 1 g | 56 μm | 12.75 | 3.98 |
| 10% PVOH, 5% polysorbate | 1 g | 88 μm | 12.09 | 1.97 |
| 50% PVOH, 0.5% polysorbate | 1 g | 111 μm | 70.11 | 5.30 |
| 50% PVOH, 0.5% polysorbate | 3 g | 310 μm | 69.11 | 5.71 |
| 50% PVOH, 5% polysorbate | 1 g | 113 μm | 53.14 | 7.40 |
| 50% PVOH, 5% polysorbate | 3 g | 358 μm | 59.94 | 3.79 |

### Example 8: Dissolution Rate of the Polymer Film (Base 25 cm²)

In order to evaluate the water solubility property of the PVOH polymer film, the dissolution rate of the film was measured for different volumes of demineralized water.

The polymer solution 30% m/v PVOH+0.5% v/v polysorbate 80 was manufactured according to Example 1 and then stored at room temperature.

1 ml of solution is deposited on a square polystyrene crystal plastic surface of 5 cm×5 cm, i.e. 25 cm². The whole is dried in a 30% ventilated oven, at 37° C. for 1 hour, allowing the polymer solution to solidify as a film.

After drying, the polymer film is removed from the plastic surface using sterile forceps. The film is then placed in a bottle containing 100, 50, 10 or 5 ml of demineralized water. The bottle is shaken by hand or vortexed to dissolve the film. The time required for complete dissolution of the film is timed.

The results are shown in the following table:

| | | | | |
|---|---|---|---|---|
| Dissolution volume (ml) | 100 | 50 | 10 | 5 |
| Average dissolution rate (min) | 1.76 | 1.61 | 2.04 | 5.36 |
| Standard deviation | 0.36 | 0.33 | 0.29 | 0.70 |

Conclusion:

The dissolution rate of the PVOH film (1 ml) is shown to be identical for a dissolution volume of 100 ml to 10 ml; i.e. 10 to 100 times more volume of demineralized water than volume of deposited film. On the other hand, the dissolution time increases for a lower dissolution volume of 5 ml (i.e. 5 times the volume of deposited film).

Finally, regardless of the dissolution volume tested, the dissolution of the PVOH film is very fast, less than 10 min even for a very small volume. This demonstrates the excellent water solubility of the PVOH film, which leaves no particles or residues after dissolution.

### Example 9: Advantage of Microorganism Sampling Using a Polymer in Liquid and/or Viscous Form According to the Present Invention

The collection of microorganisms using a polymer in liquid and/or viscous form according to the present invention was compared with collection using a polymer that was first dried before being brought into contact with the microorganism to be collected.

The polymer solution 30% m/v PVOH+0.5% v/v polysorbate 80 was manufactured according to Example 1 and then stored at room temperature.

Dry Deposit of Microorganisms:

A known quantity of Staphylococcus aureus is deposited on a glass slide (Rq≈0.5 nm). A BioBall Multishot 550 CFU (bioMérieux; item: 56019) is diluted in 500 μl of rehydration fluid (bioMérieux; item: 56021) and vortexed for 30 seconds. 50 μl of Staphylococcus aureus solution (about 50 CFU [colony forming units]) is deposited on the glass slide. The deposit is dried in a 30% ventilated oven, at 37° C. for 30 minutes.

In parallel, 50 μl of Staphylococcus aureus solution is deposited on trypticase soy agar (TSA) Petri dishes (bioMérieux; item: 43011 and 43711). These control plates are incubated at 30-35° C. for 48 hours.

Polymer Deposition:
1) Liquid form: The PVOH+polysorbate 80 polymer solution is deposited by pipetting onto the dry deposit of Staphylococcus aureus microorganisms, then spread with a sterile loop if necessary to completely cover the dry deposit. The whole is dried in a 30% ventilated oven, at 37° C. for 1 hour, allowing the polymer solution to solidify as a film.
2) Solid form: the PVOH+polysorbate 80 polymer solution is deposited by pipetting onto a glass slide and spread with a sterile loop. The solution is dried in a 30% ventilated oven, at 37° C. for 1 hour, allowing the polymer solution to solidify as a film. The film obtained is then applied for 10 seconds and with a force of 500 g on the dry deposit of Staphylococcus aureus.

Polymer Detachment and Microorganism Detection:

After drying or direct application, the polymer film is detached from the glass slide using sterile forceps. The film is then placed on a TSA Petri dish (bioMérieux; item: 43011 and 43811). After a few minutes the film is completely solubilized by virtue of the water contained in the Petri dish. The dishes are incubated at 30-35° C. for 48 hours.

Microorganism Counting and Analysis:

After 48 hours of incubation, the colonies present on the control dishes and the dishes containing the water-solubilized polymer film are counted. The recovery rate of the polymer is calculated as follows:

Recovery rate: (100×polymer TSA count)/(control TSA count)

The results are presented in the following table:

| | Average recovery | Standard |
|---|---|---|
| | rate (%) | deviation |
| Liquid form | 56.2 | 0.14 |
| Solid form | 18.7 | 0.08 |

Conclusion:

These results show that applying the water-soluble polymer solution in liquid and/or viscous form and then drying to obtain a film makes it possible to recover three times more microorganisms than the direct application of the film already in solid form.

### Example 10: Advantage of Microorganism Sampling Using a Polymer in Foam Form According to the Present Invention

The collection of microorganisms using a polymer in foam form according to the present invention was compared with a polymer solution deposited in liquid/viscous form, on a stainless-steel surface (Rq≈0.4 μm).

Dry Deposit of Microorganisms:

A known quantity of Staphylococcus aureus is deposited on a stainless-steel surface. A BioBall Multishot 550 CFU (bioMérieux; item: 56019) is diluted in 500 μl of rehydration fluid (bioMérieux; item: 56021) and vortexed for 30 seconds. 50 μl of the Staphylococcus aureus solution (about 50 CFU [colony forming units]) is deposited on the stainless-steel surface. The deposit is dried in a 30% ventilated oven, at 37° C. for 30 minutes.

Polymer Deposition:
1) Liquid form control: the polymer solution 10% m/v PVOH+0.5% v/v polysorbate 80 was manufactured according to Example 1a. This solution is deposited by pipetting on the dry deposit of Staphylococcus aureus microorganisms, then spread with a sterile loop if necessary in order to completely cover the dry deposit. The whole is dried in a 30% ventilated oven, at 37° C. for 1 hour, allowing the polymer solution to solidify as a film.
2) Foam form: The polymer foam 10% m/v PVOH+0.5% v/v polysorbate 80 was manufactured according to Example 1b. This foam is deposited on the dry deposit of Staphylococcus aureus microorganisms. The whole is dried in a 30% ventilated oven, at 37° C. for 1 hour, the foam solution goes to a liquid state before solidifying as a film.

Polymer Detachment and Microorganism Detection:

After drying, the polymer films are detached from the stainless-steel surface using sterile forceps. The film is then placed on a TSA Petri dish (bioMérieux; item: 43011 and 43811). After a few minutes the film is completely solubilized by virtue of the water contained in the Petri dish. The dishes are incubated at 30-35° C. for 48 hours.

Microorganism Counting and Analysis:

After 48 hours of incubation, the colonies present on surfaces and dishes containing the water-solubilized polymer film are counted. The recovery rate of the polymer is calculated as follows:

Recovery rate: (100×polymer TSA count)/(control TSA count)

The results are presented in the following table:

| | Average recovery | Standard |
|---|---|---|
| | rate (%) | deviation |
| Liquid solution deposit | 18 | 0.03 |
| Foam deposit | 49 | 0.12 |

Conclusion:

On a rough surface such as a stainless-steel surface, the application of a foam polymer solution makes it possible to recover about 2.5 times more microorganisms compared with an application of polymer in liquid form.

### Example 11: Influence of PVOH Concentration on the Recovery Rate

In order to study the influence of PVOH polymer concentration on the microorganism recovery rate, a range of solutions was tested: from 10 to 50% m/v PVOH with 0.5% polysorbate 80.

The solutions were manufactured according to Example 1 and then stored at room temperature.

Dry Deposit of Microorganisms:

A known quantity of Staphylococcus aureus is deposited on a glass slide. A BioBall Multishot 550 CFU (bioMérieux; item: 56019) is diluted in 500 μl of rehydration fluid (bioMérieux; item: 56021) and vortexed for 30 seconds. 50 μl of Staphylococcus aureus solution (about 50 CFU) is deposited on the glass slide. The deposit is dried in a 30% ventilated oven, at 37° C. for 30 minutes.

In parallel, 50 μl of the Staphylococcus aureus solution is deposited on TSA Petri dishes (bioMérieux; item: 43011 and 43711). These control dishes are incubated at 30-35° C. for 48 hours.

Polymer Deposition:

The PVOH+polysorbate 80 polymer solution is deposited by pipetting onto the dry deposit of Staphylococcus aureus microorganisms and then spread with a sterile loop if necessary to completely cover the dry deposit. The whole is dried in a 30% ventilated oven, at 37° C. for 1 hour, allowing the polymer solution to solidify as a film.

Polymer Detachment and Microorganism Detection:

After drying, the polymer film is detached from the glass slide with sterile forceps. The film is then placed on a TSA Petri dish (bioMérieux; item: 43011 and 43811). After a few minutes the film is completely solubilized by virtue of the water contained in the Petri dish. The dishes are incubated at 30-35° C. for 48 hours.

Microorganism Counting and Analysis:

After 48 hours of incubation, the colonies present on the control dishes and the dishes containing the water-solubilized polymer film are counted. The recovery rate of the polymer is calculated as follows:

Recovery rate: (100×polymer TSA count)/(control TSA count)

The results are shown in the following table:

| | | | | | | | | | |
|---|---|---|---|---|---|---|---|---|---|
| PVOH concentration | 10 | 15 | 20 | 25 | 30 | 35 | 40 | 45 | 50 |
| (with 0.5% polysorbate 80) | | | | | | | | | |
| in % m/v | | | | | | | | | |
| Average recovery rate (%) | 64% | 80% | 62% | 67% | 68% | 72% | 52% | 51% | 47% |
| Standard deviation | 0.06 | 0.17 | 0.14 | 0.05 | 0.06 | 0.07 | 0.12 | 0.12 | 0.08 |

Conclusion:

It has been shown that the PVOH concentration in the polymer solution does not significantly influence the recovery rate of Staphylococcus aureus, except above 35% m/v where the recovery rate tends to decrease. Below 40% m/v PVOH in solution, the Staphylococcus aureus recovery rate is comprised between 60 and 80%.

Finally, these recovery rates show a very good envelopment of the microorganisms in the film, then a very good peeling by the film and no toxicity of the PVOH+polysorbate solution.

### Example 12: Influence of Polysorbate 80 Concentration on the Recovery Rate

In order to study the influence of the polysorbate 80 concentration on the microorganism recovery rate, a range of solutions were manufactured according to Example 1: from 0 to 5% v/v polysorbate 80 with 30% m/v PVOH.

The solutions were tested using the same protocol as in Example 11.

The results are shown in the following table:

| | | | | | |
|---|---|---|---|---|---|
| Polysorbate 80 | 0 | 0.5 | 1 | 2 | 5 |
| concentration (with 30% | | | | | |
| m/v PVOH) in % v/v | | | | | |
| Average recovery rate (%) | 12% | 73% | 73% | 64% | 45% |
| Standard deviation | 0.05 | 0.12 | 0.23 | 0.19 | 0.19 |

Conclusion:

It has been shown that the polysorbate 80 concentration influences the microorganism recovery rate. In the absence of polysorbate 80, the Staphylococcus aureus recovery rate is strongly decreased. The addition of 0.5 or 1% polysorbate 80 makes it possible to obtain a recovery rate of about 70% which decreases beyond this concentration.

### Example 13: Detection on Petri Dishes

The polymer solution 30% m/v PVOH+0.5% v/v polysorbate 80 was manufactured according to Example 1 and then stored at room temperature.

The polymer solution is deposited on the surface to be sampled and then, after drying, the film is removed from the surface using sterile forceps (see Example 11 protocol).

In order to detect and count the number of microorganisms present in the film, thus initially on the surface, the film is deposited on a solid culture medium. Very quickly, the water contained in the agar solubilizes the film and it disappears completely from the surface of the culture medium. Different solid culture media can be used according to the requirements of the microorganisms to be detected.

The culture media are then incubated under the usual culture conditions. After incubation, the isolated colonies can be easily counted in order to give a quantitative result of the surface monitoring.

Tests on TSA Petri dishes (bioMérieux, item: 43811) showed that the size of colonies at 24/48/72 hours is equivalent between a direct inoculum deposit and a deposit after removal of the water-soluble polymer.

In addition, tests were carried out with a medium containing a colored indicator (Chapman medium; bioMérieux, item: 46671) and a medium containing a chromogen (SAID medium; bioMérieux, item: 419042). The results show that the staining of colonies, of the medium, and the appearance of the colonies are equivalent between a direct inoculum deposit and a deposit after removal of the water-soluble polymer.

These results demonstrate the compatibility of the water-soluble polymer with growth methods on culture medium.

### Example 14: Detection by Rapid Methods (BacT/ALERT®, Tempo®, Gene-Up®)

BacT/ALERT®:

The BacT/ALERT® technology is a kinetic microorganism detection method by colorimetry. Microorganisms present in the medium produce CO₂ during their growth phase. The CO₂ produced induces a decrease in the pH of the culture medium. This change in pH induces the colorimetric shift of a sensor composed of silicone impregnated with liquid emulsion detectors (LES), placed at the bottom of each bottle. An LED sends a light beam on the sensor.

A photodiode collects the intensity of light reflected by the sensor in the form of a reflectance unit. The reflectance units are analyzed over time.

The polymer solution 30% m/v PVOH+0.5% v/v polysorbate 80 was manufactured according to Example 1 and then stored at room temperature.

The polymer solution is deposited on a surface previously loaded with a known amount of the microorganism Staphylococcus aureus (roughly 50 CFU), as described in Example 9. The whole is dried in a 30% ventilated oven, at 37° C. for 1 hour, allowing the polymer solution to solidify as a film. After drying, the polymer film is detached from the surface using sterile forceps. The film is then diluted in 2 ml of saline solution (bioMérieux, item: 33892). After complete solubilization, the solution is withdrawn by syringe and then injected into a

BacT/ALERT® SA bottle (bioMérieux, item: 259789). In parallel, control bottles are inoculated directly with the same known quantity of the Staphylococcus aureus strain (roughly 50 CFU). The bottles are loaded onto the BacT/ALERT® VIRTUO® automated system (bioMérieux; item: 411660) for automatic kinetic reading.

The results show similar detection times and growth curves between the control bottles inoculated directly with the strain and the bottles inoculated with the solubilized polymer film. These results demonstrate the compatibility of the water-soluble polymer with a colorimetric microorganism detection method.

TEMPO®:

The Tempo technology is a method for counting microorganisms based on the most probable number (MPN) technique by analyzing series of dilutions of the initial sample. The Tempo technology miniaturizes this test on a specifically designed board. The system consists of:
A preparation station (TEMPO® FILLER) to inoculate the TEMPO® cards with the samples and the specific culture media. TEMPO® culture media allow the rapid growth of bacteria or yeast/molds and contain a fluorescent indicator. The innovative TEMPO® card integrates a 16×3 miniaturized tube MPN method.
An automated reading station (TEMPO® READER) to determine the bacterial concentration in each card and thus determine the number of microorganisms present in the initial sample.

The polymer solution 30% m/v PVOH+0.5% v/v polysorbate 80 was manufactured according to Example 1 and then stored at room temperature.

The polymer solution is deposited on a surface previously loaded with a known amount of the microorganism Staphylococcus aureus (roughly 50 CFU), as described in Example 9. The whole is dried in a 30% ventilated oven, at 37° C. for 1 hour, allowing the polymer solution to solidify as a film. After drying, the polymer film is detached from the surface using sterile forceps. The film is then placed in a TEMPO CTB bottle (bioMérieux, item: 416683). In parallel, control bottles are inoculated directly with the same known quantity of the Staphylococcus aureus strain (roughly 50 CFU). The medium contained in the bottle is rehydrated by adding 4 ml of distilled water per bottle, also allowing the polymer film to solubilize.

After rehydration and complete solubilization, the TEMPO® CTB bottles and the corresponding TEMPO® CTB cards (bioMérieux, item: 416683) are scanned on the TEMPO® FILLER station. The bottles and cards are placed on the filling rack, placed in the preparation station which will completely fill the card and then cut the straws and thus seal the card.

The cards are incubated at 30° C.±1° C. for 24 h-28 h (bacterial detection).

After incubation, the cards are placed in the TEMPO® READER reading station and read automatically. The result is displayed directly in CFU/ml, taking into account the dilution factor.

Results show equivalent bacterial counts between control bottles inoculated directly with the strain and bottles inoculated with the solubilized polymer film. These results demonstrate the compatibility of the water-soluble polymer with a fluorescence-based microorganism detection method using the MPN method.

| | | Polymer | Polymer |
|---|---|---|---|
| | Control bottles | on glass | on plastic |
| Average TEMPO ® count (in | 56 | 65 | 60 |
| CFU/ml) | | | |
| Standard deviation | 0 | 23.09 | 10.58 |

Gene UP®:

The Gene-Up® technology is a method for detecting microorganisms by real-time PCR. Following enrichment in culture medium, the DNA of the microorganisms is released by mechanical lysis and then detected by real-time PCR using FRET probes and probe fusion peaks to guarantee the specificity of the test.

The polymer solution 30% m/v PVOH+0.5% v/v polysorbate 80 was manufactured according to Example 1 and then stored at room temperature.

Different surfaces are loaded with the microorganism Listeria monocytogenes and then dried in a 30% ventilated oven at 37° C., for 30 minutes to obtain dry deposits. The polymer solution is then deposited on the microorganisms and dried in a 30% ventilated oven, at 37° C. for 1 hour, allowing the polymer solution to solidify as a film. After drying, the polymer film is removed from the surface with sterile forceps and deposited in a tube of LPT enrichment medium (bioMérieux, item: 410845). In parallel, dry deposits of microorganisms are sampled with a swab (bioMérieux, item: 70.6016) and then deposited in a tube of LPT enrichment medium. The tubes are incubated at 37° C. for 18-24 hours.

After enrichment, 20 μl of sample is deposited in a lysis tube (bioMérieux, Gene-Up® lysis kit, item: 414057). The lysis tubes are placed on a rack and shaken with the Troemner vortex at 2200 rpm for 5 minutes.

A bottle of lyophilized reagent (bioMérieux, Listeria spp. Kit: item: 414059, Listeria monocytogenes Kit: item: 414058) is reconstituted with 45 μl of reconstitution buffer. Empty PCR tubes are placed on a rack and 5 μl of PCR reagent is placed in each tube. Then 5 μl of lysed sample is added to each tube. A negative control consisting of 5 μl of control buffer is performed for each kit. The tubes are closed with stoppers, sealed and the rack is then centrifuged. The plate is placed in the Gene UP instrument.

The samples and associated detection kits are entered into the Gene UP software before starting. The results are automatically interpreted when the PCR cycle is completed. The software interprets the amplification and fusion curve data for each sample and gives a positive, negative or inhibited result.

The results show equivalent detections between the control samples and the samples collected with the polymer for all surfaces tested. In addition, all internal PCR controls are detected.

These results demonstrate the compatibility of the polymer with a detection method via the PCR technique. In particular, the polymer does not inhibit the PCR reaction.

| | Repli- | Plastic | Plastic | Glass | Glass | Negative |
|---|---|---|---|---|---|---|
| Detection kit | cate | control | polymer | control | polymer | control |
| Listeria spp. | 1 | Positive | Positive | Positive | Positive | Negative |
| | 2 | Positive | Positive | Positive | Positive | |
| Listeria | 1 | Positive | Positive | Positive | Positive | Negative |
| monocytogenes | 2 | Positive | Positive | Positive | Positive | |

### Example 15: Detection by Biochemical Method (VIDAS®)

The VIDAS® technology is a method for detecting elements (hormones, viruses, microorganisms, etc.) by immunoassay. The VIDAS® principle consists of the interaction of two elements: the cone (solid phase), whose inner surface is coated with antigens or antibodies, and the strips, composed of several wells and containing the exact quantity of reagents required for the test.

Reactions occur in the cone in two key steps:
immunological reaction, capturing the desired element;
enzymatic reaction, revealing the presence of the element sought by the ELFA (enzyme-linked fluorescent assay) technique.

The entire operation is automated: from incubation, to the washes and to the final reading. The incubation time and the number of wash cycles are optimized for each parameter to ensure optimal performance.

The polymer solution 30% m/v PVOH+0.5% v/v polysorbate 80 was manufactured according to Example 1 and then stored at room temperature.

Different surfaces are loaded with the microorganism Listeria monocytogenes and then dried in a 30% ventilated oven, at 37° C. for 30 minutes to obtain dry deposits. The polymer solution is then deposited on the microorganisms and dried in a 30% ventilated oven, at 37° C. for 1 hour, allowing the polymer solution to solidify as a film. After drying, the polymer film is removed from the surface with sterile forceps and deposited in a tube of LPT enrichment medium (bioMérieux, item: 410845). In parallel, dry deposits of microorganisms are sampled with a swab (bioMérieux, item: 70.6016) and then deposited in a tube of LPT enrichment medium. The tubes are incubated at 30° C. for 22-30 hours.

After enrichment, 500 μl of sample is deposited in the sample well of a VIDAS® UP Listeria strip (bioMérieux, item: 30126). The strip is heated for 5 minutes at 95-100° C. on the VIDAS® Heat and Go instrument and left to cool for 10 minutes.

As positive control, two standards Si are prepared by depositing 500 μl of standard Si in the sample well of a VIDAS® UP Listeria strip (bioMérieux, item: 30126). These strips are not heated.

The VIDAS® cones and strips are placed in the instrument, the samples and the associated detection parameter are entered into the software before the test is started. Results are obtained in about 62 minutes.

The results show equivalent positivity between the control samples and the samples collected with the polymer for all surfaces tested. These results demonstrate the compatibility of the water-soluble polymer with an immunoassay detection method.

### Example 16: Identification by Biochemical Method (Vitek 2)

The VITEK® 2 Compact is a microbial identification system based on miniaturized colorimetric biochemical reactions on a specifically designed card. All identification phases, from reading to recording of results, are automated. The VITEK® 2 Compact includes an extensive identification database that allows the identification of numerous microorganisms.

The polymer solution 30% m/v PVOH+0.5% v/v polysorbate 80 was manufactured according to Example 1 and then stored at room temperature.

The polymer solution is deposited on a surface previously loaded with a colony of the microorganism Kocuria kristinae. The whole is dried in a 30% ventilated oven, at 37° C. for 1 hour, allowing the polymer solution to solidify as a film. After drying, the polymer film is detached from the surface using sterile forceps. The film is placed in a hemolysis tube and then solubilized by adding 3 ml of saline solution (bioMérieux; item: 21218) for VITEK®. In parallel, a control tube containing 3 ml of saline solution is prepared to obtain 0.5 McFarland of the Kocuria kristinae strain. The tubes are loaded with a VITEK® GP card (bioMérieux; item: 21342) in the Smart Carrier Station™. Once the cassettes are loaded, the system manages the incubation and reading of each card without any further intervention. Results are obtained within 2 to 18 hours after loading.

The results show equivalent identifications (“Excellent identification”) between the control tube inoculated directly with the strain and the tube inoculated with the solubilized polymer film. These results demonstrate the compatibility of the water-soluble polymer with a colorimetric biochemical identification method.

1. A process for detecting and/or identifying at least one target microorganism present on a surface, comprising the following steps:
a) depositing on the surface, a composition comprising a film-forming water-soluble synthetic polymer, the composition being a liquid and/or viscous composition or a foam composition;
b) drying the composition to allow the formation of a polymer film,
c) removing from the surface the polymer film comprising the at least one target microorganism,
d) dissolving the polymer film with an aqueous diluent to form a solution comprising the at least one microorganism,
e) detecting and/or identifying the at least one target microorganism in all or part of the solution using at least one detection means.

2. The process as claimed in claim 1, wherein the aqueous diluent is a semi-solid culture medium, the polymer film being dissolved by contact with the semi-solid culture medium.

3. The process as claimed in claim 1, wherein the detection means is the culture medium in which the polymer film has been dissolved.

4. The process as claimed in claim 1, wherein the surface is an inert surface.

5. The process as claimed in claim 1, wherein the process comprises a step of incubating the culture medium, before or during the detection step, at a temperature and for a period of time sufficient to allow the growth of the at least one microorganism.

6. The process as claimed in claim 1, wherein the polymer is selected from polyethylene oxide, polyethylene glycol, polyvinylpyrrolidone, polyvinyl alcohol, polyacrylic acid, polyacrylamide, poly(2-hydroxypropyl methacrylamide), poly(2-ethyl-2-oxazoline).

7. The process as claimed in claim 6, wherein the polymer is polyvinyl alcohol with an average molecular weight comprised between 9 000 and 200 000 g/mol and at a concentration comprised between 2 and 60% (m/v).

8. The process as claimed in claim 1, wherein the composition comprises a surfactant.

9. The process as claimed in claim 8, wherein the surfactant is selected from sodium dodecyl sulfate, cholic acid, dicyclohexyl sulfosuccinate sodium, benzalkonium chloride, diethylene glycol, polysorbate 80, saponin, 3-(N,N-dimethyltetradecylammonio)propanesulfonate, 3-[(3-cholamidopropyl)dimethylammonio]-1-propanesulfonate hydrate.

10. The process as claimed in claim 9, wherein the surfactant s polysorbate 80 at a concentration equal to or less than 5%.
